(19) United States
(12) Patent Application Publication
(10) Pub. No.: US 2019/0059713 A1
(43) Pub. Date: Feb. 28, 2019

(54) MEDICAL SCOPE CLEANING DEVICE

(71) Applicant: Dare Surgical Design LLC, Los Angeles, CA (US)

(72) Inventors: Douglas P. Allen, Los Angeles, CA (US); Robert M. Evans, Hawthorne, CA (US)

(21) Appl. No.: 15/690,153

(22) Filed: Aug. 29, 2017

(51) Int. Cl. A61B 1/12; B08B 1/00; A61B 1/00

(52) U.S. Cl. CPC A61B 1/121; A61M 2025/0019; A61B 1/00131; B08B 1/001

(57) ABSTRACT

A medical scope cleaning device is configured to receive a distal end of a medical scope, wipe debris from the scope, apply an anti-fog/cleaning solution, and warm the scope in preparation for inserting the scope into a patient for a medical procedure. The cleaning device includes a sponge assembly disposed within a cleaning chamber. The sponge assembly includes expansion spaces. As a scope is moved through the cleaning chamber, the scope radially compresses sponges and urge at least portions of the sponges into the expansion spaces. The sponges wipe the scope and apply the solution. A heating coil warms the solution within the cleaning chamber.

### BACKGROUND

The present disclosure relates to the field of warming, cleaning, and/or defogging a medical scope such as a laparoscope or endoscope.

Medical scopes, such as laparoscopes and endoscopes, are commonly used in medical procedures, particularly in minimally-invasive procedures. Such medical scopes typically have a distally-placed lens combined with a proximally-place ocular which provides visualization within a body cavity. However, when medical scopes at cool operating room temperatures are inserted into warm body cavities, the lens will often become fogged, inhibiting vision and delaying the procedure while the device warms sufficient for the fog to subside. Also, before or during the procedure, medical scopes can come into contact with debris that may impede visualization.

Devices have been provided for applying an anti-fog solution to a medical scope lens. However, such solutions are most effective when the lens is allowed to soak in the anti-fog solution for some time, rather than simply wiping the lens with the solution. Devices are also provided to warm a scope prior to insertion into the patient in order to minimize such fogging. However, it can also take some time for a scope to warm sufficiently. Thus, the scope may be in contact with such devices for some time. If the scope, when engaged with one of such devices, is in an unstable or inconvenient position, the scope may be bumped or the like, and may fall, damaging the equipment. Additionally, pre-existing devices face challenges in effectively wiping, warming, and applying anti-fog solution to medical scopes.

### SUMMARY

There is a need in the art for a medical scope cleaning device that can apply an anti-fog/cleaning solution to a medical scope and/or warm the medical scope, and in which the device and scope are in a stable position when engaged. There is also a need in the art for a medical scope cleaning device that efficiently and effectively wipes debris from the medical scope.

In accordance with an embodiment, the present specification a medical scope cleaning device, comprising a cup assembly and a cleaning assembly. The cup assembly comprises an elongated cup body and a heating element, the cup body having a longitudinal axis, a proximal opening and a closed distal end. A cleaning chamber is defined within the cup body. The cleaning assembly is disposed within the cleaning chamber, and comprises first and second side sponges. Each of the first and second side sponges have an inner surface and an outer surface and are disposed within the cleaning chamber so that the first side sponge inner surface is on an opposite side of the axis from the second side sponge inner surface. An expansion space is defined between the outer surface of each of the first and second side sponges and an inner surface of the cup body. The first and second side sponges and the end sponge are resilient and compressible. When a tubular body of a medical scope is moved distally through the proximal opening towards the end sponge, the tubular body applies a radial force to the first and second side sponges so that the first and second side sponges are at least partially radially compressed and at least partially radially displaced into the expansion spaces.

In some embodiments, engagement portions of each of the first and second side sponges engage an inner surface of the cleaning chamber, an outer surface of each of the first and second side sponges is disposed between engagement portions, and the expansion spaces are defined between the inner surface of the cleaning chamber and the outer surfaces. Some such embodiments additionally comprise a liner interposed between the tubular body and each of the first and second side sponges. In further embodiments, at least a portion of each of the first and second side sponges is adhered to the liner. Still other embodiments additionally comprise an end sponge in the cleaning chamber, the end sponge spaced distally from a distal surface of each of the first and second side sponges.

Some embodiments additionally comprise a liner in contact with at least part of each of the first and second side sponges. In some such embodiments, the liner is anchored to the cup body so that the liner resists being pushed distally within the cleaning chamber. In further embodiments, the liner is attached to at least part of each of the first and second side sponges.

In yet additional embodiments, the liner comprises an elongated strip folded over itself, the liner having a lead-in portion in contact with a lead-in surface of the first and second side sponges, a body portion in contact with opposing inner surfaces of the first and second side sponges, the liner being on opposite sides of the axis.

An additional embodiment additionally comprises an end sponge in the cleaning chamber, the end sponge spaced distally from a distal surface of each of the first and second side sponges, the liner additionally comprising an end portion at least partially in contact with the end sponge surface.

In yet another embodiment, the liner and sponges are arranged so that when a scope tubular body is advanced distally into the cleaning chamber, the scope tubular body engages the liner and does not directly contact any of the sponges.

Still another embodiment additionally comprises a casing having a casing opening, wherein the cup assembly is supported within the casing so that the cup proximal opening is aligned with the casing opening and wherein the cup assembly axis is generally horizontal. In some such embodiments, the casing has a bottom surface, and wherein a height between the bottom surface of the casing and the cup assembly axis is about 16 mm. Still further embodiments additionally comprise an end sponge in the cleaning chamber, the end sponge spaced distally from a distal surface of each of the first and second side sponges, and an anti-fog solution is disposed with the cleaning chamber and entrained within the first side sponge, second side sponge and end sponge.

Yet another embodiment additionally comprises a case enclosing the cup assembly and defining a case opening aligned with the cup assembly proximal opening, a thermally-insulative foam also enclosed within the case and adjacent the cup assembly, the case having a dry wipe aperture spaced from the case opening, and a portion of the thermally-insulative foam extends through the dry wipe aperture.

In accordance with another embodiment, the present specification provides a method of cleaning a medical scope. The method comprises inserting a tubular body of a medical scope into a cleaning chamber generally along an axis of the cleaning chamber so that the tubular body applies a radial force urging first and second side sponges radially. The first and second side sponges are within the cleaning chamber and on opposite sides of the axis. An expansion space is defined between an outer surface of each of the first and second side sponges and an inner surface of the cleaning chamber. Each of the first and side sponges both radially compresses and is radially displaced at least partially into one of the expansion spaces by the tubular body.

Another embodiment additionally comprises a liner disposed on opposite sides of the axis, wherein the tubular body directly contacts the liner.

In yet another embodiment, the cleaning chamber is enclosed within a case so that the axis is horizontal and a distance between the cleaning chamber axis and a bottom surface of the case is the same as a radius of an ocular of the medical scope. The embodiment additionally comprises resting the case and a side edge of the ocular on a surface while a distal end of the tubular body is within the cleaning chamber, and an axis of the tubular body is coaxial with the cleaning chamber axis.

In still another embodiment, an anti-fog solution entrained within the sponges is applied to the tubular body, and additionally comprising heating the cleaning chamber.

In accordance with still another embodiment, the present specification a medical scope cleaning device also comprising a cup assembly and a cleaning assembly. The cup assembly comprises an elongated cup body and a heating element. The cup body has a longitudinal axis, a proximal opening and a closed distal end, and also defines a cleaning chamber therewithin. The cleaning assembly is disposed within the cleaning chamber, and comprises first and second side members and an end member. The end member is disposed distal of the first and second side members. The first and second side members are disposed on opposite sides of the axis and are radially compressible and resilient. A liner is on each of the first and second side members. The liner is attached to the cup body so as to resist being pushed distally. When a tubular body of a medical scope is moved distally through the proximal opening and into contact with the liner, the side members are radially compressed.

In some such embodiments, the end member comprises a resilient sponge in which an anti-fog solution is entrained.

### BRIEF DESCRIPTION OF THE DRAWINGS

FIG. 1 is a perspective view showing a laparoscope engaged with a scope cleaning device having features in accordance with an embodiment;

FIG. 2 is an exploded view of the scope cleaning device of FIG. 1;

FIG. 3 is a front view of the scope cleaning device of FIG. 1;

FIG. 4 is an exploded view of a cleaning chamber assembly of the arrangement shown in FIG. 2;

FIG. 5 is a top view of a cup assembly of the cleaning chamber assembly of FIG. 4;

FIG. 6 is an exploded view of the cup assembly of FIG. 5;

FIG. 7 is a perspective view of a cleaning assembly for use in the cup assembly of FIG. 6;

FIG. 8 is a cross-sectional view taken along lines 8-8 of FIG. 5;

FIG. 9 is a cross-sectional view taken along lines 9-9 of FIG. 5;

FIGS. 10A-C show the arrangement of FIG. 9 depicting a tubular body of a medical device being inserted into and advanced distally within the cleaning chamber;

FIG. 11 shows the arrangement of FIG. 8 but with the tubular body of a medical scope inserted in the cleaning chamber;

FIG. 12 is an exploded view of another embodiment of a scope cleaning device;

FIG. 13 is an exploded view of still another embodiment of a scope cleaning device;

FIG. 14 is an exploded view of a cleaning chamber assembly of the scope cleaning device of FIG. 13;

FIG. 15 is a side view of the cleaning chamber assembly of the scope cleaning device of FIG. 13;

FIG. 16 is a cross-sectional view taken along lines 16-16 of FIG. 15;

FIG. 17 is a cross-sectional view taken along lines 17-17 of FIG. 15;

FIG. 18A is a perspective view of a liner prior to being shaped;

FIG. 18B is a perspective view of the liner of FIG. 18A after being shaped;

FIG. 19A is a perspective view of an embodiment of a sponge assembly prior to being inserted into a cleaning chamber;

FIG. 19B is a perspective view of the sponge assembly of FIG. 19A after being shaped for insertion into a cleaning chamber; and

FIGS. 20A-C are sectional views of another embodiment of a cleaning assembly and depicting a tubular body of a medical scope being advanced through the cleaning assembly.

### DESCRIPTION

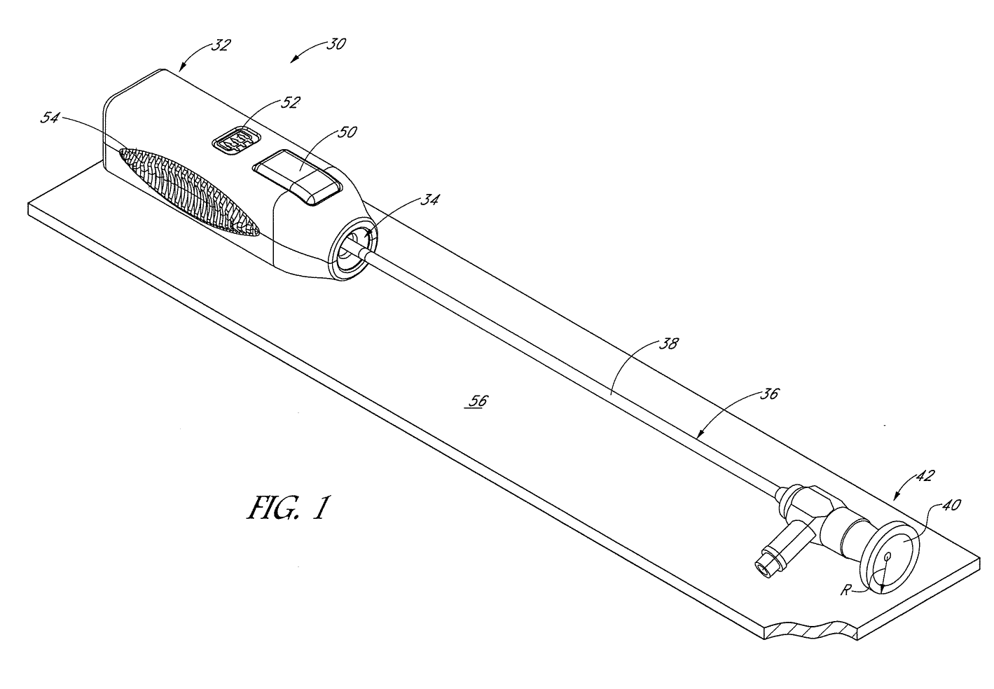

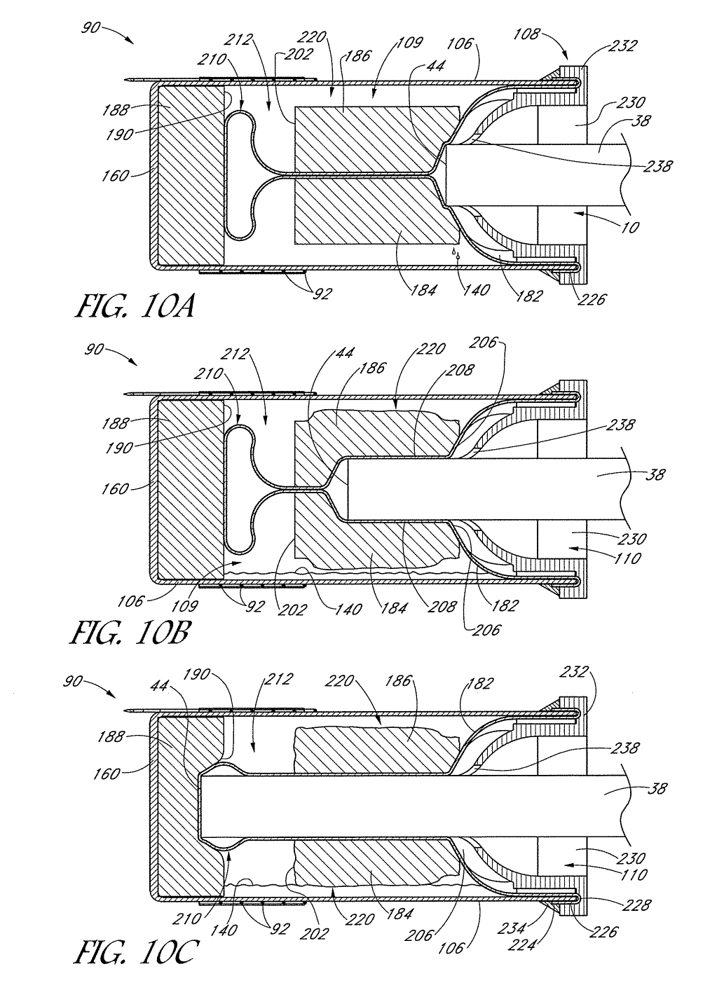

With initial reference to FIG. 1, an embodiment of a medical scope cleaner 30 comprises a case 32 having an opening 34 configured to accommodate at least part of a laparoscope 36. The illustrated laparoscope 36 comprises an elongated, substantially rigid tubular body 38. An ocular 40 is arranged at a proximal end 42 of the laparoscope 36, and is generally circular in shape, having a radius R. As shown, the tubular body 38 extends through the opening 34 and into the cleaner 30. A distal end 44 (see FIGS. 10A-C) of the laparoscope 36 typically includes a lens (not shown). As will be discussed in more detail below, the distal end 44 and lens can be warmed and cleaned within the scope cleaner 30.

The present embodiment is disclosed and configured in connection with cleaning the distal end and lens of a laparoscope 36. However, it is to be understood that the principles and structures of the embodiments discussed herein can be used to warm and clean other medical scopes, such as endoscopes.

With continued reference to FIG. 1, the illustrated medical scope cleaner 30 includes a dry wipe assembly 50 accessible from outside the case 32, and which can be used to manually wipe debris from the medical scope. A translucent window 52 is provided in the illustrated embodiment to accommodate and transmit light from an indicator light so that a user can know that the scope cleaner 30 heater function is operating. In the illustrated embodiment, a grip 54 is provided on the outer surface of the case 32. Also, as shown, the scope cleaner 30 is configured to be supported on a table or tray surface 56, preferably conveniently accessible during a medical procedure. When the distal end 44 of the tubular body 38 is received within the scope cleaner 30, as shown in FIG. 1, the illustrated laparoscope 38 is also supported upon the table or tray surface 56, with a side edge of the ocular 40 being in contact with the table 56, while the tubular body 38 is supported within the scope cleaner 30 in a generally horizontal orientation.

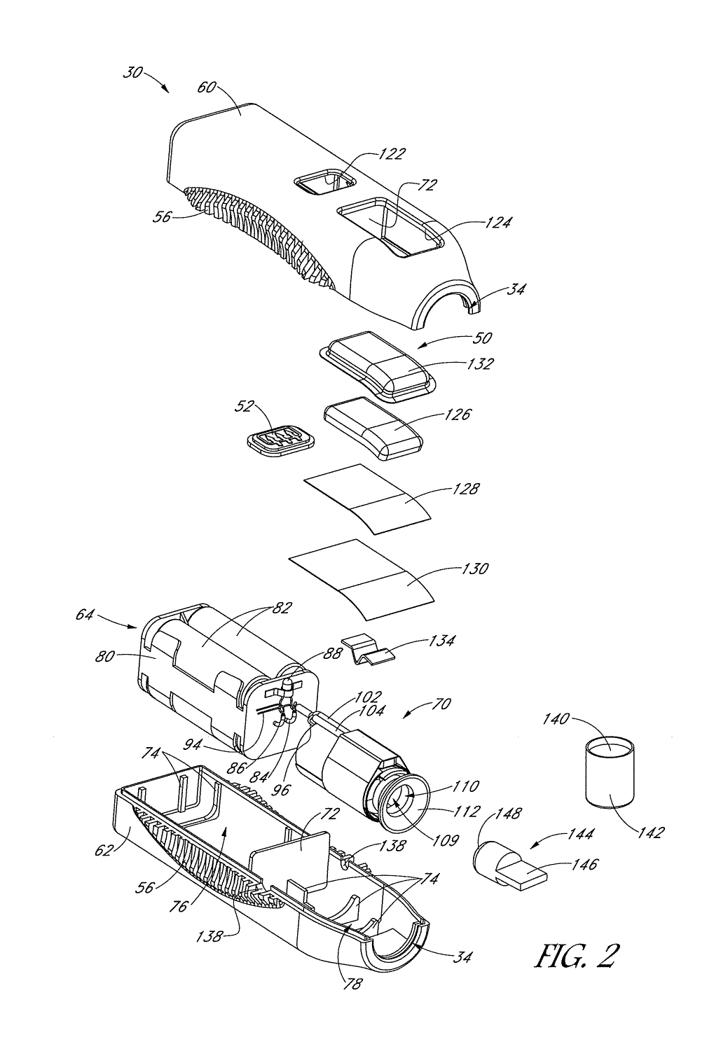

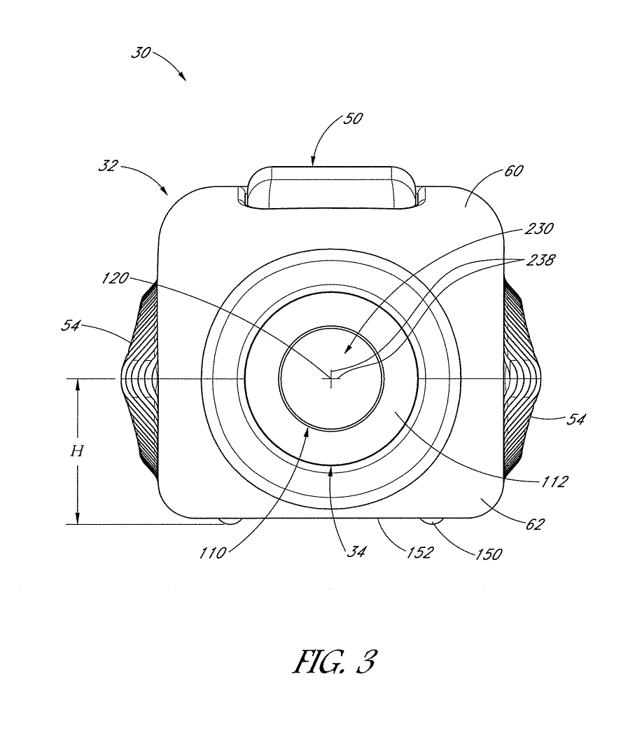

With reference next to FIGS. 2 and 3, the case 32 is made up of a top case 60 and a bottom case 62 that are configured to enclose therewithin a battery assembly 64 and a cleaning chamber assembly 70. Foam insertion holes 138 preferably are formed through opposing sides of the bottom case 62. As shown, preferably the top and bottom cases 60, 62 comprise separator walls 72 that cooperate to divide the interior of the case 32 into a battery zone 76 and a cleaner zone 78. A plurality of guides 74 are configured to engage and properly align the battery assembly 64 and cleaning chamber assembly 70 within the top and bottom cases 60, 62 so that when the cases 60, 62 are assembled, the battery assembly 64 fits generally within the battery zone 76, and the cleaning chamber assembly 70 fits generally within the cleaner zone 78. When assembled, the top and bottom cases 60, 62 combine to define the case opening 34.

In the illustrated embodiment, the battery assembly 64 comprises a battery case 80 that is configured to accommodate multiple battery cells 82. The case 80 includes circuitry configured to direct electrical output of the battery cells 82 between first and second battery wires 84, 86. An LED lamp 88 is supported on a proximal wall of the battery case 80 and is electrically connected to the battery wires 84, 86 so that the lamp will illuminate when current flows therethrough, providing an indication that a warming function of the scope cleaner 30 is operating. Of course, it is contemplated that various configurations of a battery assembly 64 can be employed, including embodiments employing rechargeable or non-rechargeable cells configured in accordance with various shapes and structures.

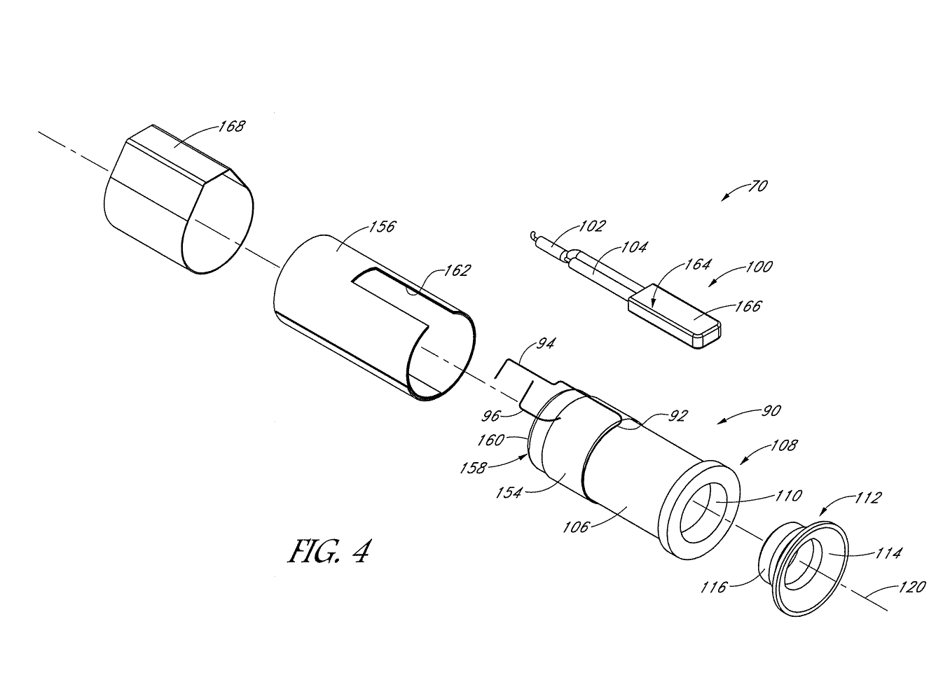

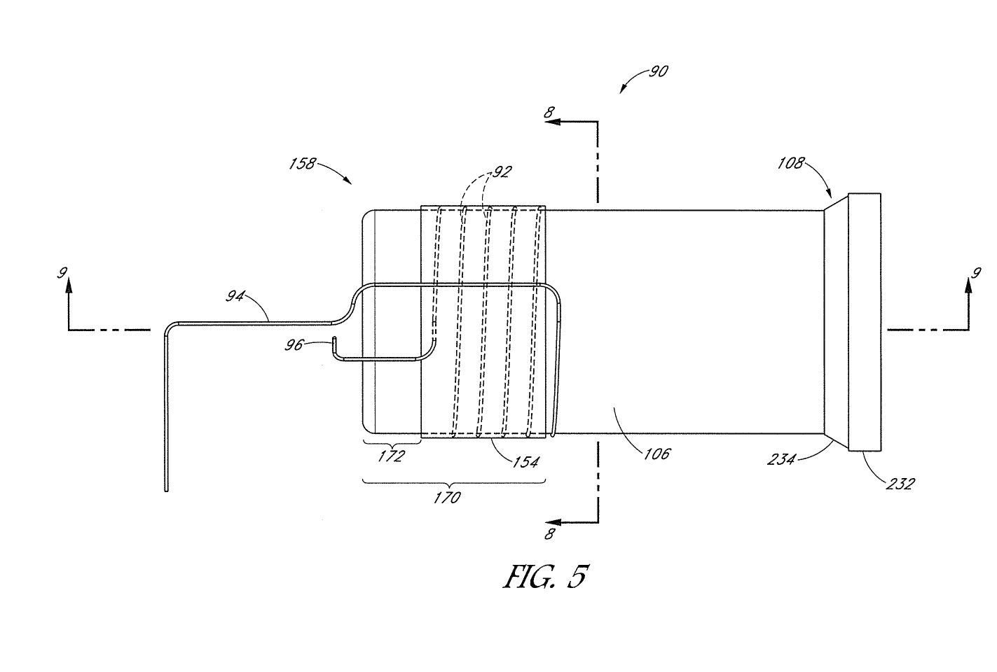

With additional reference to FIGS. 4 and 5, the cleaning chamber assembly 70 comprises a cup assembly 90 having a heating coil 92 with first and second leads 94, 96. A thermostat 100 has a first thermostat wire 102 that is attached to the first battery wire 84 and a second thermostat wire 104 that is attached to the second lead 96 of the heating coil 92. The first lead 94 of the heating coil 92 is configured to be selectively attachable to the second battery wire 86 in order to complete the circuit and supply electrical energy from the battery assembly 64 to the heating coil 92 to activate the warming function. The first lead 94 and second battery wire 86 can be selectively connected in any desired manner. For example, in one embodiment a switch may be button-actuated to selectively complete the circuit; in another embodiment a nonconductive pull strip (not shown) may be disposed between the first lead 94 and second battery wire 86, or between any of the batter cells 82 and a corresponding circuit contact of the battery case 80 so that when the nonconductive strip is removed the circuit is completed.

With continued reference to FIGS. 2-5, the cup assembly 90 comprises an elongated cup body 106 that defines a cleaning chamber 109 therewithin. At a proximal end 108 of the cup assembly 90, an access opening 110 is defined through which a distal end 44 of the laparoscope tubular body 38 may pass to enter the cleaning chamber 109. A funnel 112 comprises a tapered funnel opening 114 at its proximal end, which funnel opening 114 leads to a tubular funnel body 116 that extends distally from the funnel opening 116. In the illustrated embodiment, the funnel body 116 extends into and is fit in the access opening 110. The access opening 110 and funnel opening 114 are both aligned with an axis 120 of the cleaning chamber assembly 70. The cup assembly 90 and cleaning chamber 109 also lie along the axis 120.

With specific reference again to FIG. 2, in the illustrated embodiment, the top case 60 includes a window aperture 122 into which the translucent window 52 is fit. Preferably, the window aperture 122 is arranged so as to be generally aligned with the LED lamp 88 so that illumination of the lamp 88 can be seen through the window 52.

The top case 60 also defines a dry wipe aperture 124 into which the dry wipe assembly 50 is fit. In the illustrated embodiment, the dry wipe assembly 50 comprises a sponge pad 126 adhered via tape 128 to a support 130. A cloth cover 132, which preferably is formed of a textile material such as a microfiber, is disposed over the sponge pad 126. A spacer 134 is attached to the bottom of the support 130 so as to depend downwardly therefrom. The dry wipe assembly 50 is inserted and received within the dry wipe assembly aperture 124. In some embodiments, the spacer 98 depends into the cleaner zone 78 sufficient to engage a portion of the cleaning chamber assembly 70. In the illustrated embodiment, the sponge pad 126 is resilient so that when the dry wipe assembly 50 is engaged by a medical scope, the sponge pad 126 compresses in order to expand and improve contact between the cloth cover 132 and the medical scope.

To assemble the scope warmer 30, window 52 is placed within the window aperture 122 and the dry wipe assembly 50 is assembled and placed within the dry wipe aperture 124. The battery assembly 64 and cleaning chamber assembly 70 are placed within and between the top and bottom cases 60, 62, and aligned properly therein via engagement with the guides 74 so that the funnel 112 is aligned with the case opening 34 as the top and bottom cases 60, 62 are put into engagement and closed upon one another as shown in FIGS. 1-3. Once assembled, an expanding foam preferably is injected through the foam insertion holes 138, substantially filling the cleaner zone 78 and surrounding the cleaning chamber assembly 70. Preferably the injected foam has advantageous heat insulation properties.

An anti-fog/cleaning solution 140, depicted in FIG. 2 in a solution cup 142, can be inserted through the funnel 114 and access opening 110 into the cleaning chamber. A stopper 144 preferably includes a tab 146 configured to enable manipulation by a user, and a plug 148 that can be fit into the access opening 110 as desired to selectively plug the access opening 110 and block anti-fog,/cleaning solution 140 from inadvertently draining from the cleaning chamber assembly 70. Most preferably the plug 148 is put in place for transport and storage in order to resist potential spilling or evaporation of anti-fog/cleaning solution.

With specific reference to FIG. 3, in the illustrated embodiment, feet 150 depend from a bottom surface 152 of the bottom case 62. A height H is defined from the bottom surface 152 or bottom of the feet 150 to the axis 120. Preferably, the height H is substantially the same as the radius R of the ocular 40 of the laparoscope 36 being used with the scope warmer cleaner 30 (see also FIG. 1). Currently, typical laparoscope oculars are circular and have a standardized radius R of 16 mm, and thus preferably the height H is about 16 mm. As such, when the scope cleaner 30 and laparoscope 36 are resting upon the table surface 56, with the tubular body 38 inserted through the access opening 110 and into the cleaning chamber 109, the laparoscope ocular 40 will rest on its edge also on the surface 56, as depicted in FIG. 1. In this arrangement, the tubular body 38 of the laparoscope 36 is maintained in a horizontal position, coaxial with the axis 120 of the cleaning chamber assembly 70. In this manner, optimal positioning of the distal end of the laparoscope body 38 can be achieved and maintained within the cleaning chamber assembly 70. Also, the laparoscope 36 and scope cleaner 30 are stable in this arrangement and can be left in this position with little fear of being bumped or spontaneously falling. In additional embodiments, the height H is within 10%, and in still additional embodiments is within about 25%, of the radius R. Also, it is contemplated that the standardized ocular radius may change in the future or that the generally circular shape of today's standard oculars may also change. Preferably, however, the height H will be selected so that the tubular body 38 is maintained generally coaxial with the axis 120 when the distal end of the tubular body 38 is inserted through the access opening 110 into the cleaning chamber 109, and the side of the ocular 40 and the scope cleaner 30 rest upon the same table surface 56.

With specific reference next to FIGS. 2 and 4, in the illustrated embodiment, the coil 92, which comprises a resistance wire wrapped several times about a portion of the cup body 106, is in turn covered with a circumferential shrink wrap 154. A tube of insulative tape 156 is configured to fit over the cup body 106, extending generally from the open proximal end 108 of the cup body 106 to a distal end 158 of the cup body 106. Notably, the distal end 158 of the cup body 106 preferably is closed, having a distal wall 160. As such, the cleaning chamber 109 within the cup body 106 encloses and contains the anti-fog/cleaning solution, and structure for cleaning and applying the solution to a scope pushed into the cleaning chamber 109. The illustrated insulative tape 156 preferably includes an elongated slot 162.

The thermostat 100 comprises an elongated thermostat body 164 having a top surface 166. The slot 162 preferably is generally complementary to the shape of the thermostat body 164 so that the thermostat body 164 fits through the slot 162 so as to directly contact the outer surface of the cup body 106. Another tubular layer of shrink wrap 168 circumferentially surrounds the cup body 106 and thermostat body 164 along at least the length of the thermostat body 164. In a preferred embodiment, the thermostat body 164 comprises electronic controls, such as sensors and control circuitry, sufficient to control power delivery to the heating coil 92 to effect warming of the cup assembly 90 so that the cleaning chamber 109 of the cup body 106 is kept within a desired range of temperature during the warming operation of the scope cleaner 30.

With reference next to FIGS. 4-5, a distal portion 170 of the cup body 106 is defined as the distal half or distal third of the cup body 106. Preferably, the heating coil 92 is wrapped about the cup body 106 several times in at least a part of the distal portion 170. In the illustrated embodiment, the coil 92 is spaced a distance 172 from the distal end 158 of the cup body 106.

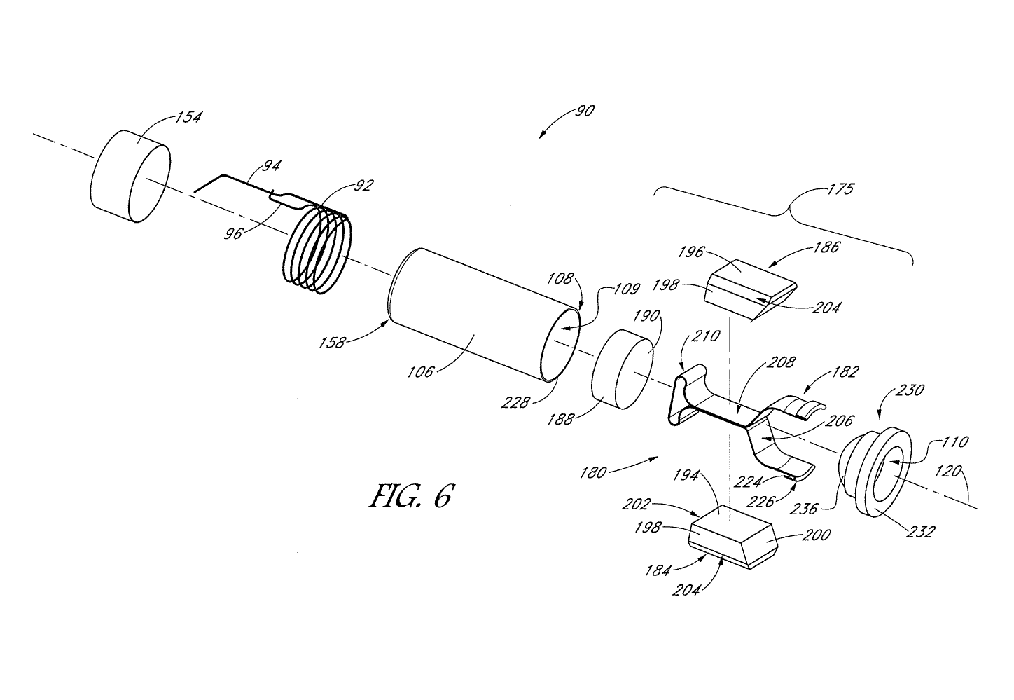

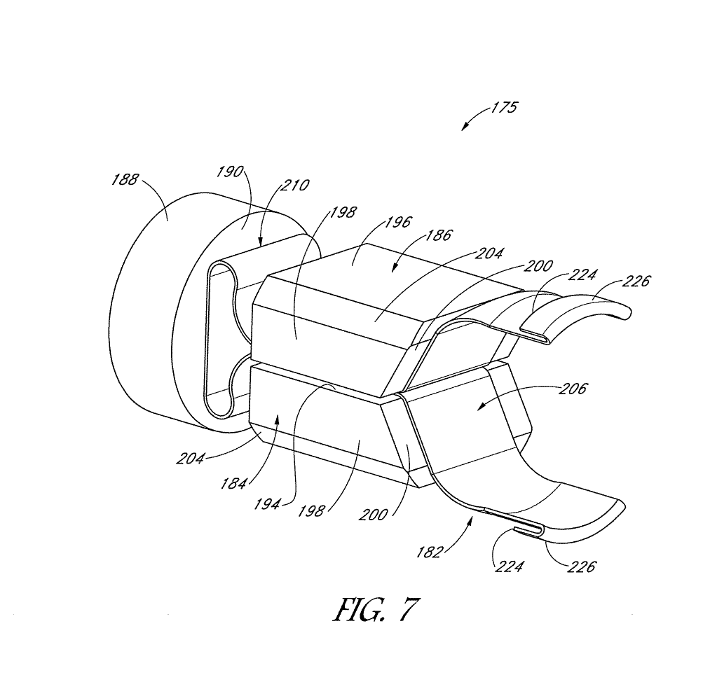

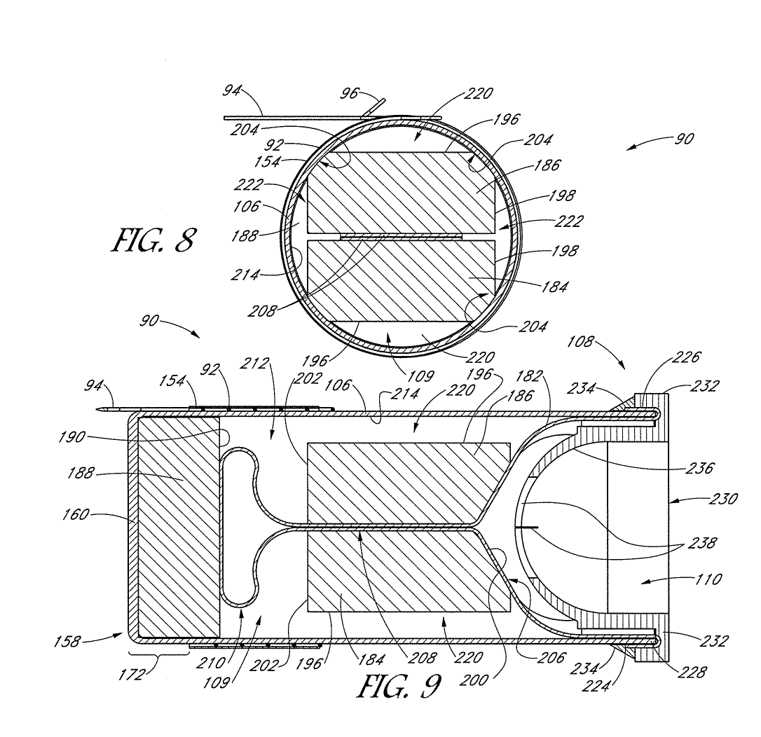

With reference next to FIGS. 6-9, a cleaning assembly 175 is fit into the cleaning chamber 109. The cleaning assembly 175 comprises a sponge assembly 180 and a liner 182. In the illustrated embodiment, the sponge assembly 180 comprises opposing first and second side sponges 184, 186 and an end sponge 188 that are formed separately from one another. The end sponge 188 is arranged against the distal wall 160 and has an end sponge surface 190. Preferably, a thickness of the end sponge 188 is greater than distance 172 so that at least a portion of the end sponge 188 overlaps the coil 92, and the end sponge surface 190 is proximal of a distal end of the coil 92.

In the illustrated embodiment, the first and second side sponges 184, 186 are substantially identical, each having an inner surface 194, an outer surface 196, opposing side surfaces 198, a lead-in surface 200 and a distal surface 202. In the illustrated embodiment, each lead-in surface 200 is tapered so as to be at an acute angle relative to the axis 120. Elongated engagement portions 204 are disposed between each outer surface 196 and its adjacent side surface 198. The engagement portions 204 provide a transition between adjacent outer and side surfaces 196, 198. In the illustrated embodiment, the engagement portions 204 are generally flat and at a 45° angle relative to both the adjacent outer and side surfaces 196, 198.

The liner 182 preferably comprises a single elongated strip of textile material, such as a microfiber cloth, folded strategically and engaged with the sponge assembly 180 so as to define a lead-in portion 206, body portion 208, and end portion 210. In the illustrated embodiment, the liner 182 is folded symmetrically about the axis 120 so that each of the lead-in portion 206, body portion 208 and end portion 210 includes two layers of the liner 182 that are mirror images of one another and on opposite sides of the axis 120.

In the illustrated embodiment, the lead-in portion 206 engages and rests upon the lead-in surfaces 200 of the side sponges 184, 186, and inner surfaces 194 engage the body portion 208. When the cleaning assembly 175 is positioned within the cup body 106, an indicator space 212 is defined between the end sponge surface 190 and the distal surfaces 202 of the side sponges 184, 186. The end portion 210 of the liner 182 is disposed within the indicator space 212 and partially in contact with the end sponge surface 190. As shown, preferably the end portion 210 of the liner 182 spreads out in the indicator space 212 so as to overlap itself and present substantial slack. In the illustrated embodiment, the side sponge distal surfaces 202 are distal of the proximal end of the heating coil 92 so that the heating coil 92 at least partially overlaps the side sponges 184, 186.

In a preferred embodiment, the lead-in portion 206 of the liner 182 is adhered to the lead-in surface 200 of the side sponges 184, 186. In some embodiments, this is the only portion of the liner that is adhered to the sponge assembly. In some such embodiments, only part of the lead-in portion 206 is adhered to the lead-in surface 200. However, the liner is connected sufficiently so that the surfaces 200 generally move with the liner 182. In other embodiments, at least part of the body portion 208 of the liner 182 is also adhered to the inner surface 194 of its respective side sponge. In still further embodiments at least part of the end portion 210 is adhered to the end sponge surface 190. In additional embodiments, one or more, or all of these sections of the liner can be adhered to respective portions of the sponge assembly 180. In the illustrated embodiment, a width of the liner 182 is less than a width of the side sponge inner surfaces 194. In other embodiments, such widths may be substantially the same.

As best shown in FIG. 8, when the cleaning assembly 175 is installed within the cup body 106, the engagement portions 204 of the side sponges 184, 186 engage the inner surface 214 of the cup body 106. An outer expansion space 220 is disposed between each side sponge outer surface 196 and the cup body inner surface 214, and a side expansion space 222 is disposed between each side sponge side surface 198 and the cup body inner surface 214. Preferably, the engagement portions 204 are compressed somewhat against the inner surface 214 so that the side sponges 184, 186 are securely held in place.

It is contemplated that other embodiments may employ side sponges having shapes that differ from those of the illustrated embodiment. For example, in another embodiment, sponge outer and side surfaces may meet at a corner at substantially a right angle. The engagement portion would entail such corner and portions of the side and outer surfaces, and the sponge in the engagement portion would be compressed into engagement with the cup body inner surface. Still other embodiments may employ sponges with some measure of curvature and varying angles in their side and outer surfaces, and engagement portions. In still further embodiments the side sponges do not engage the inner surface of the cup body.

With reference again to FIGS. 5-9, each of the ends 224 of the liner 182 preferably are turned backwards to form an overlap 226. As shown, when the liner 182 is placed in the cup body 106, the overlaps 226 are placed about and over a proximal edge 228 of the cup body 106 so that the ends 224 are on the outside of the cup body 106. In the illustrated embodiment, a dome valve 130 having a flange 232 is placed atop the cup body 106 so that the flange 232 sandwiches the overlap 226 between the proximal edge 228 of the cup body 106 and the flange 232. Preferably, a layer or bead 234 of adhesive, such as an epoxy, is applied to the cup body 106, liner ends 224 and flange 232 about the circumference of the cup body 106. As such, the liner 182 is securely held in place and will not be pushed distally into the cup body 106 when a distally-directed force is applied to the liner 182. Preferably, the bead 234 of adhesive is applied so as to seal the flange 232 and the ends 224 of the liner 182 to the cup body 106 and to block the ends 224 of the liner 182 from exposure to the environment so as to prevent leakage of solution from the chamber 109 and prevent wicking of solution to the environment via the liner 182.

In the illustrated embodiment, the dome valve 230 defines the access opening 110. A dome portion 236 of the dome valve 230 extends distally into the cup body 106 and preferably is made of an elastomeric material. In the illustrated embodiment, a plurality of valve slits 238 are formed through the dome portion 236 so as to enable a laparoscope tubular body 38 to deform dome portion 236 and pass through the valve slits 238 and into contact with the cleaning assembly 175 in the cleaning chamber 109 within the cup body 106.

With reference next to FIGS. 1 and 10A, to use the scope cleaner 30, the user inserts the distal end 44 of a laparoscope 86 tubular body 38 through the opening 34. The distal end 44 proceeds distally through the access opening 110 and into engagement with the dome valve 230, deflecting the slits 238, passing through the valve and into engagement with the lead-in portion 206 of the liner 182 within the cleaning chamber 109. As discussed above, the lead-in portion 206, 200 of the liner 182 and associated side sponges 184, 186 is disposed at an acute angle relative to the axis 120. The advancing distal end 44 thus applies a force to the liner 182 that has both axial and radial components. Since the liner 182 is prevented from being pushed distally, advancement of the distal end 44 of the tubular body 38 urges the body portions 208 of the liner 182 to move radially and separate from one another, initiating radial compression of the side sponges 184, 186. In this embodiment, the sponges are not directly contacted by the tubular body 38. Thus, the side sponges are not in friction-bearing contact with the tubular body 38, and compression or displacement of the side sponges 184, 186 in an axial direction is minimal or substantially zero when the tubular body 38 is advanced distally. Instead, the sponges are compressed and displaced radially as the tubular body is inserted into the cleaning chamber.

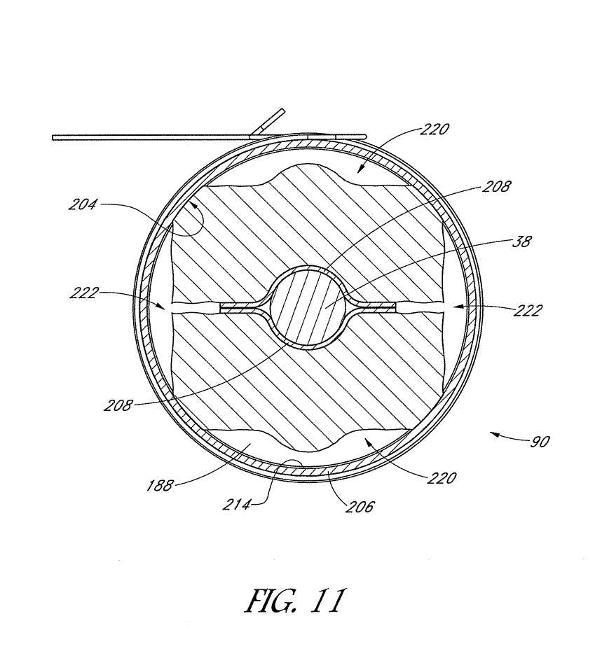

With additional reference to FIG. 10B, as the tubular body 38 continues to be advanced distally into the cup assembly 90, the body portions 208 continue to be pulled radially apart, and the side sponges 184, 186 continue to be radially compressed and displaced, while the liner 182 engages and wipes the tubular body 38. Radial forces exerted by the sponges and resistance to compression urge the liner 182 against the tubular body 38, enhancing engagement. As shown in FIGS. 10B and 11, the sponges 184, 186 are partially compressed and partially displaced into the outer expansion spaces 220 and side expansion spaces 222 (see FIG. 8). Since anti-fog/cleaning solution 140 preferably is entrained within the sponges, some of the solution 140 may be pushed out of the sponges as they compress. Some of that solution is pushed through the wetted liner 182 and into contact with the tubular body 38, and some such solution 140 can be expected to at least partially accumulate along the bottom of the cleaning chamber 109.

As discussed above, continued advancement of the tubular body 38 radially compresses and displaces the sponges 184, 186, and the sponges exert forces in resistance. A user pushing the tubular body 38 into the scope cleaner 30 works against such resistance forces and feels them as a tactile insertion resistance force. However, once the distal end 44 passes the distal surfaces 202 and enters the indicator space 212, compression of the sponges temporarily stops, and the tactile resistance to advancement felt by the user suddenly decreases. The user thus knows that the distal end 44 has passed by the side sponges 184, 186. With continued advancement of the tubular body 38, the distal end 44 will next engage the end portion 210 of the liner at the end sponge surface 190, and began compressing the end sponge 188. As such, resistance to advancement will increase again, and the user may know that the distal end 44, which incorporates the lens, is engaged with the end sponge 188, and the tubular body 38 is fully inserted into the scope cleaner 30. As also discussed above, the end portion 210 of the liner 182 has substantial slack. At least some of that slack is taken up as the tubular body 38 extends through the indicator space 212 and engages and at least partially compresses the end sponge 188. In some embodiments, the user can rotate the tubular body 38 back and forth about its axis to help clean, wet, and wipe the tubular body 38.

When the warming function is activated, the heating coil 92 heats the contents of the cup body 106, and particularly the distal portion 170 of the cup body 106. As discussed above, the cleaning chamber assembly 70 is encased within an insulative foam, and heat will accumulate throughout the cleaning chamber 109. As such, the sponge assembly 175 and anti-fog/cleaning solution 140 entrained therein is warmed. This warmth is transferred to the lens at the distal end 44 of the laparoscope tubular body 38, as well as to a portion of the tubular body 38 near the distal end 44. Preferably, about the distal-most inch of the tubular body 38 is warmed and wiped when inserted into the scope cleaner 30. In some embodiments, the tubular body 38 is inserted into the scope cleaner 30 while preparing for a procedure, and the laparoscope 36 is left in place in the position as shown in FIG. 1 for some time while the distal end 44 is warmed. Warming and cleaning the laparoscope 38 enhances its performance when later inserted into the patient.

In the illustrated embodiment, opposing lengths of the liner 182 in the body portion 208 are substantially planar and straddle opposing sides of the axis 120. More particularly, lengths of the body portion 208 lie upon the inner surfaces 194 of the first and second side sponges 184, 186. Such inner surfaces 194 are planar in the illustrated embodiment. More particularly, in the illustrated embodiment, the inner surfaces 194 each lie in a horizontal plane. It is contemplated that the first and second side sponges 184, 186 can be arranged differently if desired. For example, in another embodiment the inner surfaces 194 may each lie in a substantially vertical plane.

Various materials can be used in the cleaning assembly 175. For example, the liner 182 preferably is made of a textile material particularly effective at wiping debris and the like from the laparoscope 36. Preferably the liner is wettable and readily communicates fluid, such as the anti-fog/cleaning solution, onto the laparoscope body 38. It is also preferred that the liner be made of a material from which small fibers will not dislodge when wiping the tubular body 38, as such fibers may interfere with the lens. In a preferred embodiment, the liner comprises a microfiber lens cleaning cloth made of an 80%/20% polyester—polyamide blend. In other embodiments, the liner comprises a microfiber polyester—polyamide blend lens cleaning cloth made up of at least 70% polyester.

Various materials can also be used for the sponge assembly 180. In this specification, the term “sponge” refers to a material that is flexible and will elastically recoil. Preferably, a “sponge” also has advantageous wicking and liquid entrainment properties. In some preferred embodiments, the sponge comprises an open or closed cell foam having both elastic recoil and wicking ability. Most preferably, an open cell foam is employed having a density of about 1.0-1.8 and more preferably about 1.2 lb./in.³, and a resiliency of about 35% to 45%, and more preferably about 35%. Examples of some acceptable foams that are currently available are known as acoustic, medical, and charcoal regular.

Foam having acceptable properties, combined with the expansion spaces, enables the scope warmer 30 to accommodate scopes of various diametrical sizes. For example, a small scope having a diameter of about 3 mm will modestly compress the side sponges 184, 186 when it is inserted. Resisting such compression, the resilient side sponges will urge the liner 182 into engagement with the tubular body 38 with sufficient force so that the liner wipes the scope effectively. A large scope having, for example, a 10 mm diameter, will certainly be engaged with sufficient force to wipe the tubular body 38. Due to the expansion spaces, however, the insertion force required to push such a large-diameter scope into the cleaning chamber 109 between the side sponges 184, 186 is reduced so as to still be within a reasonable range that is much less than if the expansion spaces were not provided. Also, preferably the sponges wick anti-fog/cleaning solution sufficiently that solution is entrained throughout substantially the entire sponge assembly, and every part of the tubular body 38 that contacts the cleaning assembly 175 is wetted by the solution.

Various solutions may be used to clean the laparoscope 38 and treat the lens to resist fogging. In a preferred embodiment, the anti-fog/cleaning solution comprises 85% distilled water, 13% isopropyl alcohol and 2% surfactant. Of course, it is contemplated that other solutions may be acceptably employed as desired. Further, in some embodiments, the cup body 106 may be completely filled with solution so that there is substantially no empty space within the cleaning chamber. In other embodiments, the volume of solution within the cup body 106 is such that all or most of the solution is entrained within the sponge assembly. Still further embodiments may find a middle ground between such embodiments.

Additional embodiments may contemplate varying structures and configurations of the cleaning assembly. For example, in another embodiment, a sponge assembly may include a foam end sponge, but employ side sponges, also suitably referred to as side members, made of a plastic or even metal spring arrangement configured to urge the liner against the scope body as it passes therebetween. Such springs can be radially compressible as the tubular body 38 is inserted into the cleaning chamber. In still further embodiments, springs employed as side members may include a layer of foam. Some such embodiments may also employ a liner, while other embodiments may not employ a liner or may have a liner material formed unitarily with other materials in the side member.

In still further embodiments, the cleaning assembly 175 can be provided without a liner 182. Instead, the laparoscope tubular body 138 can directly engage the sponge assembly 180. In yet additional embodiments, a liner may be provided over only portions of the sponges. For example, in some embodiments, a liner may be provided only in the lead-in portion, or may be provided in the lead-in portion and part of the body portion.

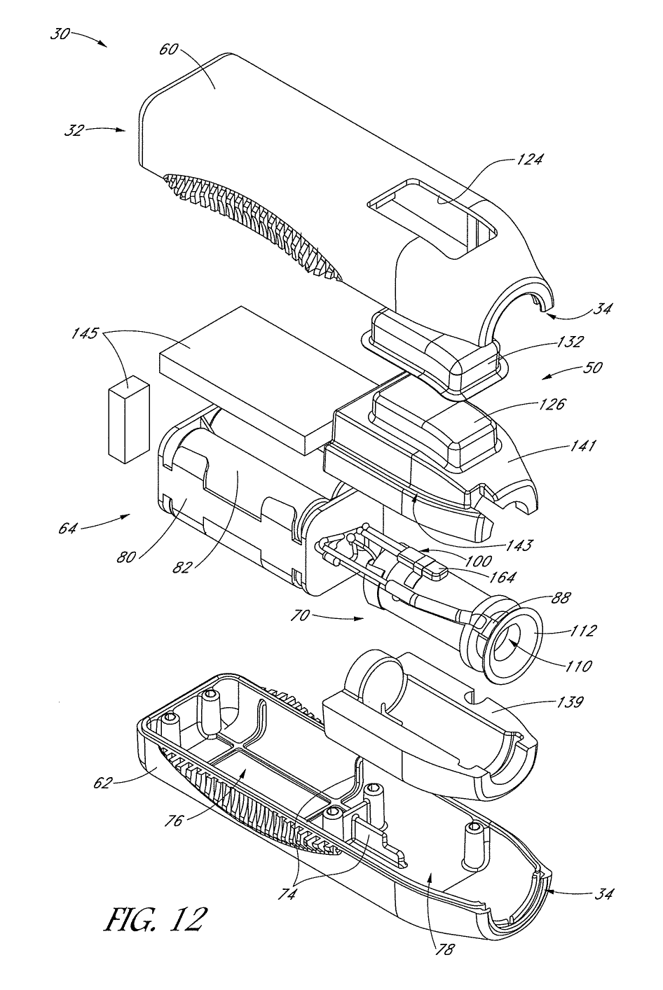

With reference next to FIG. 12, another embodiment of a medical scope cleaner 30 also includes a case 32 made up of a top case 60 and a bottom case 62 that cooperate to enclose a cleaning chamber assembly 70 and a battery assembly 64 therewithin. The illustrated embodiment is similar in many ways to the embodiment discussed above in connection with FIGS. 1-11. However, in the illustrated embodiment, rather than employing a coil, a ceramic heating element (not shown) is placed adjacent the cup body distal wall, and the thermostat 100 is located more distally along the cleaning chamber assembly 70, preferably so that the thermostat body 164 is generally aligned with the indicator space within the cleaning chamber 109. Additionally, in the illustrated embodiment, the funnel 112 is translucent, and the LED lamp 88 is placed adjacent the translucent funnel 112 so that, when illuminated, the LED 88 illuminates the translucent funnel 112, providing both an indicator that the warming function is operational and illuminating the cleaning chamber opening 110.

With continued reference to FIG. 12, a lower molded foam insert 139 is placed within the bottom case 62 and is shaped complementarily to the cleaning chamber assembly 70 and the bottom case 62 so that the lower portion of the cleaning chamber assembly 70 is received into and supported by the lower molded foam insert 139. Similarly, an upper molded foam insert 141 is shaped complementarily to the cleaning chamber assembly 70 and top case 62 so that it engages and supports the upper portion of the cleaning chamber assembly 70 within the case 32. In the illustrated embodiment, the upper molded foam insert 141 comprises an elongated wire slot 143 extending generally longitudinally and configured to receive electric wires supplying power from the battery assembly 64 to the LED lamp 88 adjacent the funnel 112.

A sponge pad 126 is defined by a protruding portion of the upper molded foam insert 141, and is configured to extend through the dry wipe aperture 124. Preferably, the sponge pad 126 is unitarily formed with the rest of the upper molded foam insert 141. Also, preferably a microfiber cloth cover 132 is fit over the sponge pad 126. More specifically, the cloth cover 132 is fit over the sponge pad 126 so that when the cloth cover 132 and sponge pad 126 are pushed through the dry wipe aperture 124 edges of the cloth cover 132 are sandwiched between the body of the upper molded foam insert 141 and the top cover 60, holding the cover 132 tightly in place atop the sponge pad 126 so as to define the dry wipe assembly 50.

With continued reference to FIG. 12, additional foam inserts 145 can be placed within the cases 60, 62 so that the battery assembly 64 and cleaning chamber assembly 70 are held securely in place, properly insulated, and/or to resist vibration.

Various foam materials can be used for the upper and lower molded foam inserts 141, 139. In a preferred embodiment, the upper and lower foam inserts 141, 139 are formed of a resilient, thermally insulative closed cell foam such as polyethylene.

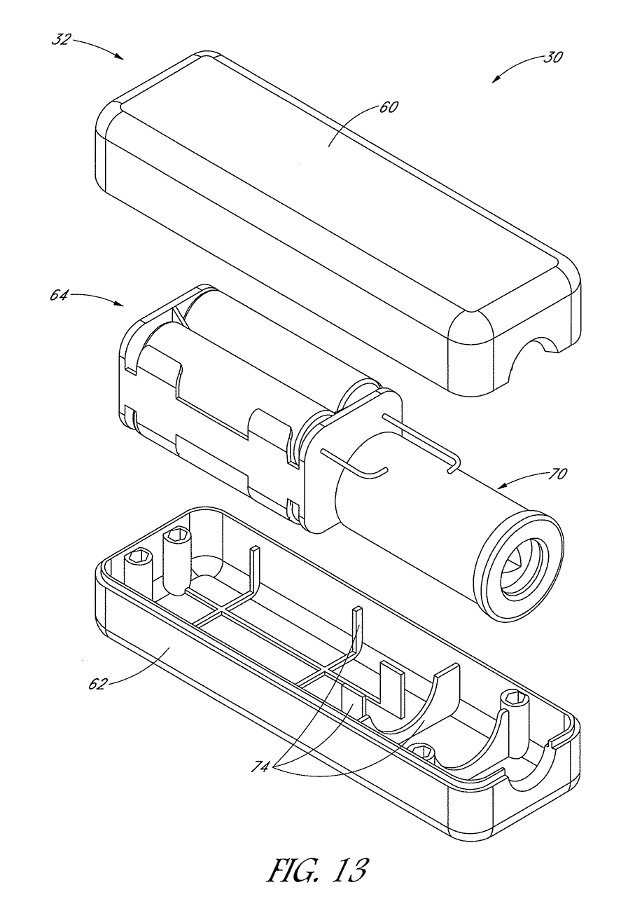

With reference next to FIG. 13, still another embodiment of a medical scope warmer 30 also includes a case 32 made up of a top case 60 and a bottom case 62. As shown, the shape and configuration of the case 32 differ more substantially from the embodiments described above. Nevertheless, in the illustrated embodiment, the cleaning chamber assembly 70 and the battery assembly 64, which powers the warming operation of the cleaning chamber assembly 70, can be enclosed within the case 32. The illustrated top and bottom cases 60, 62 each include a plurality of guides 74 that engage and ensure proper positioning of the battery assembly 64 and cleaning chamber assembly 70 as the top and bottom cases 60, 62 are engaged and closed upon one another.

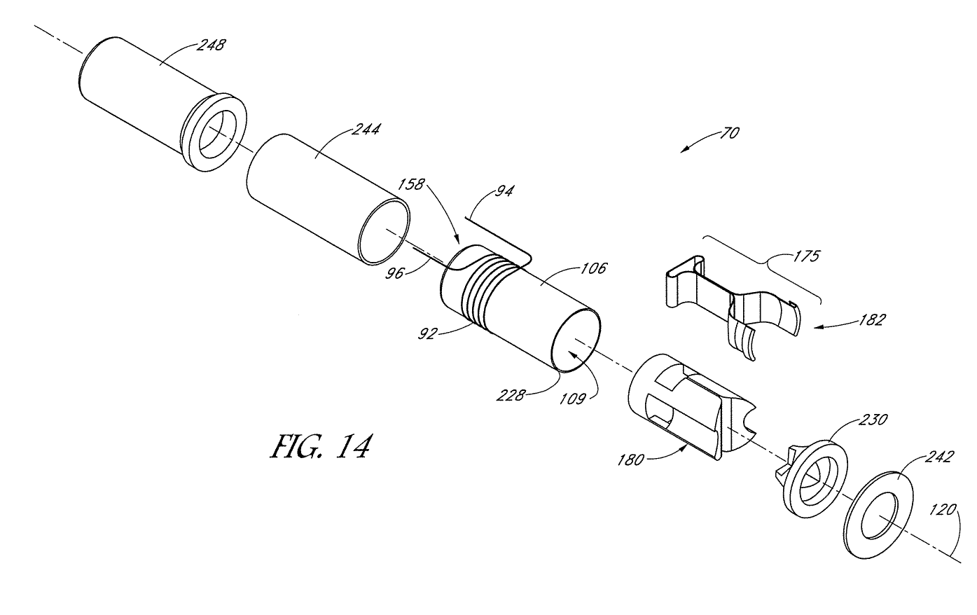

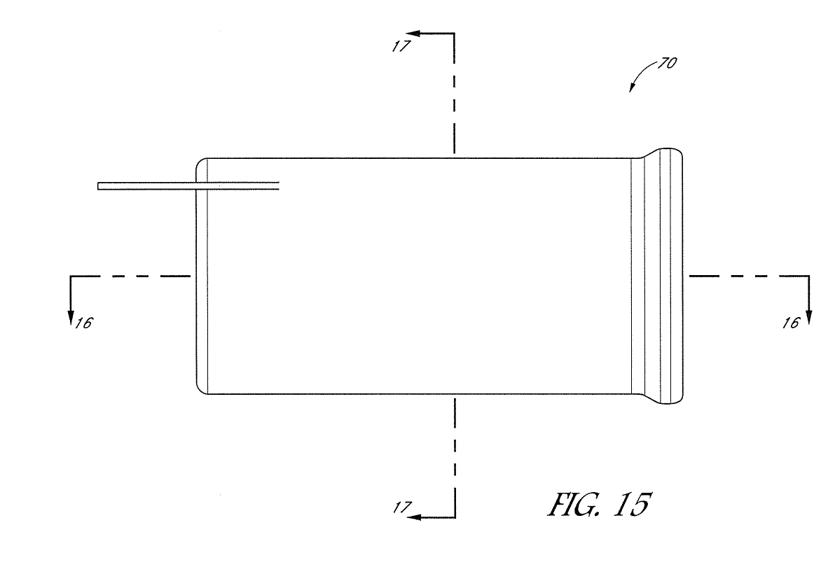

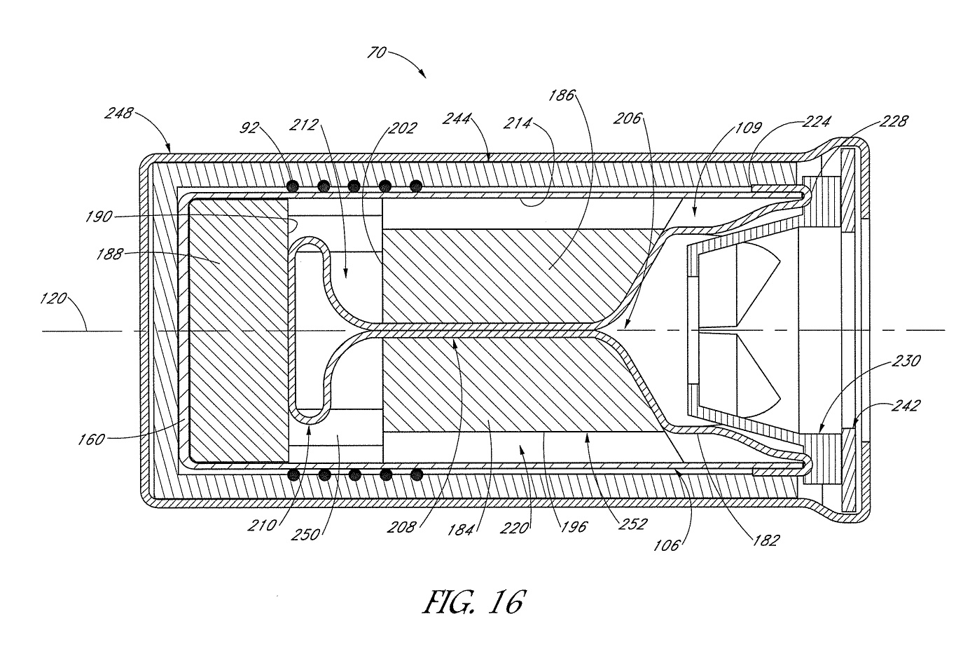

With reference next to FIGS. 14-17, in the illustrated embodiment, the cleaning chamber assembly 70 also comprises an elongated cup body 106 having a proximal edge 228 opposite a distal end 158. The cup body 106 is open adjacent its proximal edge 228 and defines a cleaning chamber 109 therewithin. A heating coil 92 is wrapped several times around an outer surface of the cup body 106, and includes first and second leads 94, 96 configured to receive electrical energy from the battery assembly 64.

A cleaning assembly 175 comprising a sponge assembly 180 and a liner 182 is fit within the cleaning chamber 109 of the cup body 106. A valve 230 includes a flange 232 that engages the proximal edge 228, and a portion of the valve 230 extends into the cup body 106. In the illustrated embodiment, a washer 242 engages a proximal surface of the valve flange 232. An insulative wrap 244 fits over the cup body 106 and heater coil 92. The illustrated insulative wrap 244 is shaped to generally complement the outer surface of the cup body 106, and extends substantially from adjacent the proximal edge 228 to and around the distal end 158 of the cup body 106. A shrink wrap 248 is then fit over the insulative wrap 244 and cup body 106. In the illustrated embodiment, the shrink wrap 248 substantially encases the entire cleaning chamber assembly 70, including the washer 242, valve 230 cup body 106 and insulative wrap 244, completely enclosing the distal end 158 of the cup body 106, and partially enclosing at least the washer 42 by extending radially inwardly proximal of the proximal edge 228 of the cup body 106. The leads 94, 96, however, preferably extend through and out of the insulative wrap 244 and shrink wrap 248.

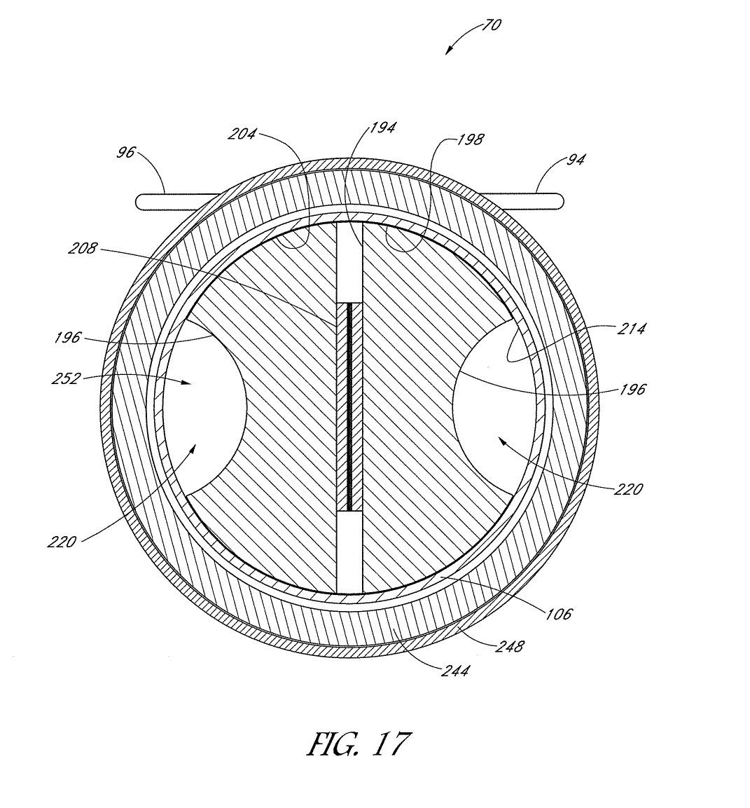

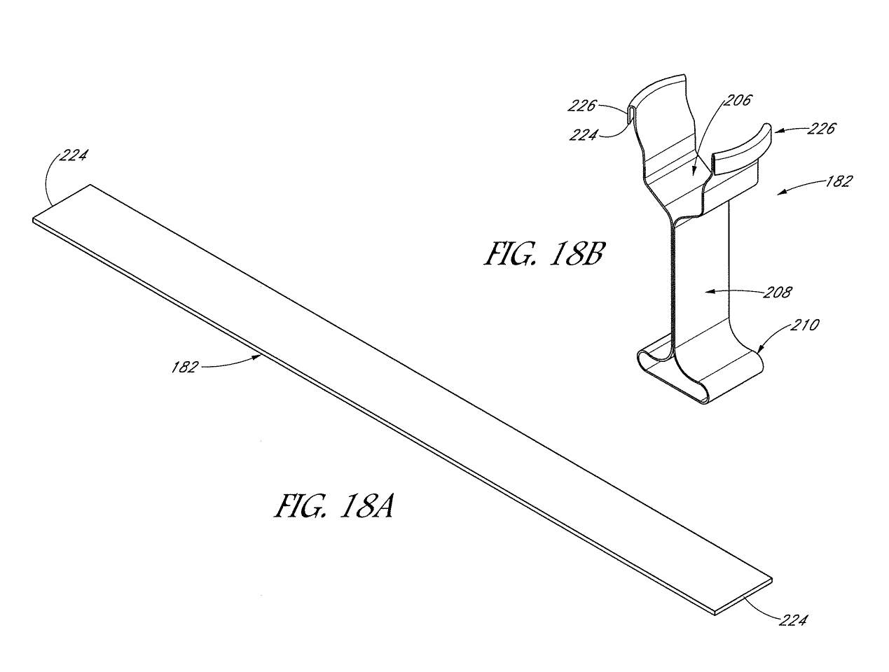

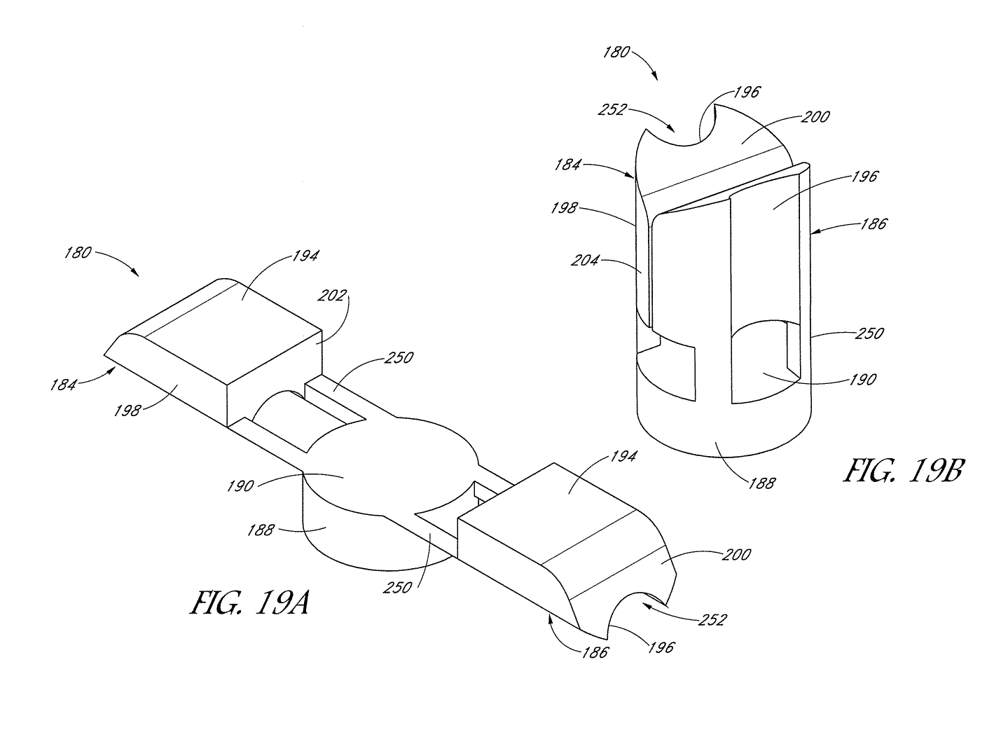

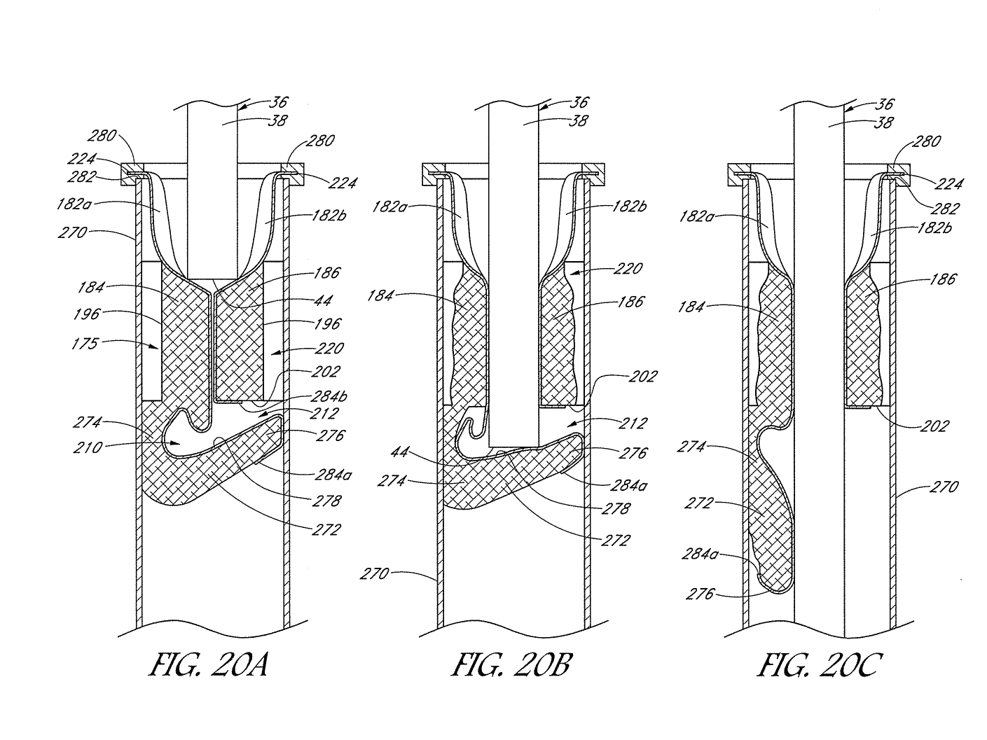

With additional reference to FIGS. 18 and 19, the cleaning assembly 175 in the illustrated embodiment is similar to embodiments as discussed above. For example, as shown in FIGS. 18A-B, the liner 182 comprises an elongated strip of textile material, preferably a microfiber cloth, folded over itself to form two mirror-image halves. As shown, the liner 182, when shaped, includes a lead-in portion 206, body portion 208 and end portion 210. The opposing ends 224 of the liner 182 are folded backward the over themselves to form an overlap 226.

With specific reference to FIGS. 13, 16 and 19A-B, the illustrated sponge assembly 180 includes a first side sponge 184 and a second side sponge 186 that are also substantially mirror images of one another. Each of the side sponges 184 preferably comprises a planar inner surface 194, an arcuate outer surface 196, opposing arcuate side surfaces 198, planar distal surfaces 202 and lead-in surfaces 200. The lead-in surfaces 200 have a planar portion that is set at an acute angle relative to the axis 120. A curving portion of the lead-in surfaces 200 curves to transition from the planar portion to the planar inner surface 194. A disk shaped end sponge 188 has a surface 190 that is spaced from the distal surfaces 202 of the first and second side sponges 184, 186. A plurality of elongated connectors 250 extend from the end sponge 188 to the side sponges 184, 186. In the illustrated embodiment, two connectors 250 connect to each side sponge 184, 186. It is to be understood, however, that other embodiments may employ more or less such connectors.

As best shown in FIG. 19A, the illustrated sponge assembly 180 is formed as a single piece. More particularly, in some embodiments, the sponge assembly 180 is molded in the shape shown in FIG. 19A, in which the inner surfaces 194 of the side sponges 184, 186 are generally coplanar and parallel to the end sponge surface 190. During assembly, and with reference to FIGS. 13-19, the sponge assembly 180 is deformed to move the side sponges 184, 186 so that the body portion 208 of the liner 182 is engaged between the inner surfaces 194, and the lead-in portion 206 rests upon the lead-in portion 200, thus forming the cleaning assembly 175. The assembled cleaning assembly 175 is then fit into the cleaning chamber 109. In some embodiments, a portion of the liner 182 can be connected to the sponge assembly via an adhesive.

The illustrated sponge assembly 180 preferably is formed of a resilient material, such as one or more of the materials discussed above in connection with other embodiments. As noted above, the illustrated sponge assembly 180 is deformed to fit within the cleaning chamber 109. As such, the resilient sponge assembly 180 is biased towards its original configuration and thus exerts a biasing force against the inner surface 214 of the cup body 106. Such a biasing force leads to firm engagement between an engagement portion 204 of the side sponges 184, 186 and the cup body inner surface 214. In the illustrated embodiment, the arcuate side surfaces 198 make up the engagement portion 204 and preferably are curved substantially complementary to the inner surface 214, which is circular in cross-section. The curved outer surfaces 196 define a cutout 252 relative to the curvature of the side surfaces 198. As such, expansion spaces 220 are defined between the outer surfaces 196 and the inner surface 214 of the cup body 106.

In operation, electricity provided by the battery assembly 64 to the heating coil 92 warms the contents of the cleaning chamber 109, which includes the cleaning assembly 175 and, preferably, an anti-fog/cleaning solution. As a user inserts the distal end of a medical scope through the valve 230 and distally into the cleaning chamber 109, the scope distal end contacts the liner 182, and the side sponges 184, 186 are radially compressed as well as radially displaced into the expansion spaces 220. When the distal end of the scope passes the distal surfaces 202 it moves through the indicator space 212, providing tactile feedback informing the user that the scope has passed between the side sponges and that the next increase in insertion resistance indicates that the distal end/lens of the scope has engaged the end sponge 188. Engagement with the end sponge 188 enables wiping and application of solution to the lens in the distal end 44.

It is to be understood that cleaning assemblies employing structure and attributes discussed in connection with the above embodiments can be employed to clean medical scopes in other environments and structures. For example, with reference next to FIG. 20A-C, another embodiment of a cleaning assembly 175 can be employed inside, for example, a trocar 270 rather than within a separate scope cleaner. In the illustrated exemplary embodiment, a sponge assembly comprising first and second side sponges 184, 186 is inserted into a portion of a tubular trocar 270 so that the engagement portions 204 of the sponges engage an inner surface of the trocar 270, and expansion spaces 220 are provided between portions of an outer surface 196 of each sponge and the inner surface of the trocar 270.

In the illustrated embodiment, a wiper sponge 272 is spaced distally from the distal surfaces 202 of the side sponges 184, 186, and an indicator space 212 is defined between the distal surfaces 202 and the wiper sponge 272. The wiper sponge 272 is part of the first side sponge 184, and a base 274 of the wiper sponge 272 depends from the distal surface 202 of the first side sponge 184. Preferably, the base 274 engages a portion of the inner surface of the trocar 270, and the wiper sponge 272 traverses across the diameter of the trocar so that a tip 276 of the wiper sponge 272 engages the inner surface on the opposite side of the trocar 270. In the illustrated embodiment an upper surface 278 of the wiper sponge 272 is angled generally upwardly from the base 274 to the tip 276.

With continued reference to FIGS. 19A-C, preferably the liner 182 comprises first and second liner portions 182a, 182b that together define a lead-in portion 206, body portion and body portion 208. Preferably the liner is anchored so that when the distal end 44 of a laparoscope 36 pushes distally against the liner, the liner will not be pushed distally within the trocar. In the illustrated embodiment, ends 224 of each liner portion 182a, b are encased and captured within an anchor flange 280, which in turn rests upon a proximal end 282 of the trocar 270. As such, the anchor flange 280 prevents the liner 182 from being pushed distally into the trocar 270.

In the illustrated embodiment, the first liner portion 182a extends distally beyond the first side sponge distal surface 202, and an end portion 210 is defined distal of the distal surface 202. The end portion 210 preferably includes a slack portion, and extends across the wiper sponge 272 upper surface 278 and around the tip 276, terminating at a distal end 284a. In the illustrated embodiment, the first liner portion 182a is adhered to the wiper sponge 272 at least at or adjacent the base 274 and at the distal end 284a. Preferably, the distal end 284b of the second liner portion 182b is adhered to the distal surface 202 of the second side sponge 186.

With reference next to FIG. 19B, as the tubular body 38 of the laparoscope 36 is advanced through the cleaning assembly, the side sponges 184, 186 preferably are compressed and displaced in a manner as discussed above. Further, as the distal end 44 clears the distal surfaces 202, the user is given tactile feedback, via a reduction in insertion resistance, making the user aware that the next structure to be encountered will be the wiper sponge 272, which will wipe the distal end 44 and lens of the laparoscope 36. As the distal end 44 engages the wiper sponge 272, the wiper sponge 272 presses and deflects to engage the lens. Continued distal advancement of the tubular body 38 further compresses and displaces the wiper sponge 272 which rotates about its base 274 so that the sponge 272 and liner 182a is drawn transversely across the distal end 44, thus wiping the lens. As the tubular body 38 continues to be advanced, the wiper sponge 272 is rotated out of the way of the laparoscope 36, as shown in FIG. 19C.

Of course, it is to be understood that the configuration depicted in FIGS. 19A-C is only exemplary. There are several different configurations of trocars, many of which include additional structure at or near the proximal end or edge of the trocar. As such, additional embodiments are contemplated to use various types of structures to anchor the liner, as well as various shapes and structures for the wiper sponge. Also, in some embodiments, rather than being placed directly within the trocar, a cleaning assembly can, in some embodiments, be contained in a separate tubular structure that can be releasably connected to the proximal end of the trocar when desired, such as after the trocar has been placed within the patient's body.

The embodiments discussed above have disclosed structures with substantial specificity. This has provided a good context for disclosing and discussing inventive subject matter. However, it is to be understood that other embodiments may employ different specific structural shapes and interactions.

Although inventive subject matter has been disclosed in the context of certain preferred or illustrated embodiments and examples, it will be understood by those skilled in the art that the inventive subject matter extends beyond the specifically disclosed embodiments to other alternative embodiments and/or uses of the invention and obvious modifications and equivalents thereof In addition, while a number of variations of the disclosed embodiments have been shown and described in detail, other modifications, which are within the scope of the inventive subject matter, will be readily apparent to those of skill in the art based upon this disclosure. It is also contemplated that various combinations or subcombinations of the specific features and aspects of the disclosed embodiments may be made and still fall within the scope of the inventive subject matter. Accordingly, it should be understood that various features and aspects of the disclosed embodiments can be combined with or substituted for one another in order to form varying modes of the disclosed inventive subject matter. Thus, it is intended that the scope of the inventive subject matter herein disclosed should not be limited by the particular disclosed embodiments described above, but should be determined only by a fair reading of the claims that follow.

What is claimed is:

1. A medical scope cleaning device, comprising:
a cup assembly comprising an elongated cup body and a heating element, the cup body having a longitudinal axis, a proximal opening and a closed distal end, and defining a cleaning chamber therewithin; and
a cleaning assembly disposed within the cleaning chamber, the cleaning assembly comprising first and second side sponges, each of the first and second side sponges having an inner surface and an outer surface and being disposed within the cleaning chamber so that the first side sponge inner surface is on an opposite side of the axis from the second side sponge inner surface, an expansion space being defined between the outer surface of each of the first and second side sponges and an inner surface of the cup body, the first and second side sponges and the end sponge being resilient and compressible;
wherein when a tubular body of a medical scope is moved distally through the proximal opening towards the end sponge, the tubular body applies a radial force to the first and second side sponges so that the first and second side sponges are at least partially radially compressed and at least partially radially displaced into the expansion spaces.

2. A medical scope cleaning device as in claim 1, wherein engagement portions of each of the first and second side sponges engage an inner surface of the cleaning chamber, an outer surface of each of the first and second side sponges is disposed between engagement portions, and the expansion spaces are defined between the inner surface of the cleaning chamber and the outer surfaces.

3. A medical scope cleaning device as in claim 2 additionally comprising a liner interposed between the tubular body and each of the first and second side sponges.

4. A medical scope cleaning device as in claim 3, wherein at least a portion of each of the first and second side sponges is adhered to the liner.

5. A medical scope cleaning device as in claim 2 additionally comprising an end sponge in the cleaning chamber, the end sponge spaced distally from a distal surface of each of the first and second side sponges.

6. A medical scope cleaning device as in claim 1 additionally comprising a liner in contact with at least part of each of the first and second side sponges.

7. A medical scope cleaning device as in claim 6, wherein the liner is anchored to the cup body so that the liner resists being pushed distally within the cleaning chamber.

8. A medical scope cleaning device as in claim 7, wherein the liner is attached to at least part of each of the first and second side sponges.

9. A medical scope cleaning device as in claim 8, wherein the liner comprises an elongated strip folded over itself, the liner having a lead-in portion in contact with a lead-in surface of the first and second side sponges, a body portion in contact with opposing inner surfaces of the first and second side sponges, the liner being on opposite sides of the axis.

10. A medical scope cleaning device as in claim 9 additionally comprising an end sponge in the cleaning chamber, the end sponge spaced distally from a distal surface of each of the first and second side sponges, the liner additionally comprising an end portion at least partially in contact with the end sponge surface.

11. A medical scope cleaning device as in claim 9, wherein the liner and sponges are arranged so that when a scope tubular body is advanced distally into the cleaning chamber, the scope tubular body engages the liner and does not directly contact any of the sponges.

12. A medical scope cleaning device as in claim 1 additionally comprising a casing having a casing opening, wherein the cup assembly is supported within the casing so that the cup proximal opening is aligned with the casing opening and wherein the cup assembly axis is generally horizontal.

13. A medical scope cleaning device as in claim 12, wherein the casing has a bottom surface, and wherein a height between the bottom surface of the casing and the cup assembly axis is about 16 mm.

14. A medical scope cleaning device as in claim 13 additionally comprising an end sponge in the cleaning chamber, the end sponge spaced distally from a distal surface of each of the first and second side sponges, and an anti-fog solution is disposed with the cleaning chamber and entrained within the first side sponge, second side sponge and end sponge.

15. A medical scope cleaning device as in claim 1 additionally comprising a case enclosing the cup assembly and defining a case opening aligned with the cup assembly proximal opening, a thermally-insulative foam also enclosed within the case and adjacent the cup assembly, the case having a dry wipe aperture spaced from the case opening, and a portion of the thermally-insulative foam extends through the dry wipe aperture.

16. A method of cleaning a medical scope, comprising:
inserting a tubular body of a medical scope into a cleaning chamber generally along an axis of the cleaning chamber so that the tubular body applies a radial force urging first and second side sponges radially, the first and second side sponges being within the cleaning chamber and on opposite sides of the axis, an expansion space being defined between an outer surface of each of the first and second side sponges and an inner surface of the cleaning chamber;
wherein each of the first and side sponges both radially compresses and is radially displaced at least partially into one of the expansion spaces by the tubular body.

17. A method as in claim 16 additionally comprising a liner disposed on opposite sides of the axis, wherein the tubular body directly contacts the liner.

18. A method as in claim 16, wherein the cleaning chamber is enclosed within a case so that the axis is horizontal and a distance between the cleaning chamber axis and a bottom surface of the case is the same as a radius of an ocular of the medical scope, and additionally comprising resting the case and a side edge of the ocular on a surface while a distal end of the tubular body is within the cleaning chamber, and an axis of the tubular body is coaxial with the cleaning chamber axis.

19. A method as in claim 18, wherein an anti-fog solution entrained within the sponges is applied to the tubular body, and additionally comprising heating the cleaning chamber.

20. A medical scope cleaning device, comprising:
a cup assembly comprising an elongated cup body and a heating element, the cup body having a longitudinal axis, a proximal opening and a closed distal end, and defining a cleaning chamber therewithin;
a cleaning assembly disposed within the cleaning chamber, the cleaning assembly comprising first and second side members and an end member, the end member disposed distal of the first and second side members, the first and second side members being disposed on opposite sides of the axis and being radially compressible and resilient; and
a liner on each of the first and second side members, the liner attached to the cup body so as to resist being pushed distally;
wherein when a tubular body of a medical scope is moved distally through the proximal opening and into contact with the liner, the side members are radially compressed.

21. A medical scope cleaning device as in claim 20, wherein the end member comprises a resilient sponge in which an anti-fog solution is entrained.
